(19) United States
(12) Patent Application Publication
(10) Pub. No.: US 2019/0008843 A1
(43) Pub. Date: Jan. 10, 2019

(54) PHARMACEUTICAL COMPOSITIONS FOR ON DEMAND ANTICOAGULANT THERAPY

(71) Applicant: Kenox Pharmaceuticals Inc, South Plainfield, NJ (US)

(72) Inventors: Sitaram VELAGA, Luleå (SE); Vijay JOGUPARTHI, Luleå (SE)

(21) Appl. No.: 16/118,535

(22) Filed: Aug. 31, 2018

Related U.S. Application Data
(63) Continuation of application No. PCT/SE2017/050179, filed on Feb. 27, 2017.

(30) Foreign Application Priority Data
Mar. 2, 2016 (SE) 1650278-3

(51) Int. Cl. A61K 31/4439; A61K 9/70; A61K 47/10; A61K 47/38; A61K 47/22; A61K 47/26

(52) U.S. Cl. CPC A61K 31/4439; A61K 9/7007; A61K 47/26; A61K 47/38; A61K 47/22; A61K 47/10

(57) ABSTRACT

A pharmaceutical composition for administration of Dabigatran zetexilate through the mucous membranes of the oral cavity, characterized in that the composition comprises a therapeutically effective amount of Dabigatran etexilate and a solubilizer for Dabigatran etixilate selected from at least one of a water soluble derivative of tocopherol and a polymeric derivative of sorbitan. The composition may be in the form of solid oral film. The composition may be suitable for dropwise administration. A method of manufacturing the film composition is also included.

### FIELD OF INVENTION

This invention relates to pharmaceutical compositions for treating, Deep Vein Thrombosis (DVT) or Pulmonary Embolism by anticoagulant therapy. By administering pharmaceutical formulations through membranes in the oral cavity according to the invention, an improvement in efficacy and safety of orally administered direct thrombin inhibitors is enabled.

### BACKGROUND OF INVENTION

Thrombosis is the underlying cause of many cardiovascular disorders such as Unstable Angina, Myocardial Infarction and Ischemic Stroke. Anticoagulant therapy with small molecular weight heparins and Vitamin K antagonists was the main stay of pharmacotherapy in treatment and management of thromboembolic diseases for several decades before the recent development of Direct thrombin inhibitors (DTIs) acting as anticoagulants by directly inhibiting the enzyme thrombin. FDA approved DTIs include bivalirudin, lepirudin, argatroban, desirudin and Dabigatran. Dabigatran, in the form of the currently marketed dosage form, Pradaxa® is the only orally administered DTI, however it exhibits relatively poor oral bioavailability (3-7%) and is associated with several adverse effects, most notably gastrointestinal disorders and bleeding disorders. In order to improve its oral absorption, Dabigatran is manufactured as the prodrug Dabigatran etexilate, which is a double prodrug of Dabigatran with low aqueous solubility at pH≥3. Dabigatran etexilate is classified as a BCS class II compound due to its high intrinsic passive permeability and low aqueous solubility. Following peroral administration, Dabigatran etexilate is rapidly absorbed in the intestinal tract and hydrolyzed by esterases to the active moiety Dabigatran. In spite of rapid absorption, its poor oral bioavailability is indicative of low solubility in the gastrointestinal milieu which is confirmed by clinical studies, suggesting substantial amounts of unabsorbed drug residing in the gastrointestinal tract following oral administration that may be the cause of the documented gastrointestinal disorders. Therefore pharmaceutical formulations and methods of administration Dabigatran that can reduce the gastrointestinal adverse events, while attaining systemically necessary drug exposure at lower doses of drug and reducing the variability in exposure associated with co-administration of other drugs or foods, are needed.

Developing oral formulations of Dabigatran etexilate is challenging, because under physiological conditions relevant to oral absorption (pH 4.5-7.4), it is virtually insoluble. In addition to its poor solubility, Dabigatran etexilate undergoes rapid degradation in aqueous media. The hydrolysis of the prodrug results in the formation of Dabigatran which has reduced permeability by virtue of it being predominantly ionized under physiological conditions. Dabigatran etexilate subjected to acidic conditions in the stomach (pH<2) and mildly acidic to neutral conditions (pH 4.5-7.4) in the small and large intestine is highly unstable at both acidic and neutral conditions which also may contribute to the low oral bioavailability in vivo.

It would be desirable to provide pharmaceutical compositions of Dabigatran etexilate that reduce residual unabsorbed highly potent drug in the gastrointestinal tract, reduce its clinical dosage while achieving the same therapeutic plasma levels and efficacy. By achieving the same therapeutic levels with reduced dosage of the drug, it is possible to reduce the side-effects since it is well understood in the field of art that a higher dose poses a greater risk of side-effects when compared to lower doses for many classes of therapeutic drugs.

EP 2722033 and US 2008039391 disclose buccal or sublingual dose forms of Dabigatran etexilate. Examples of oral formulations of dabigatran etexilate with solubility enhancers are disclosed in WO2013/17594; US 2015/366813 and CN 20141821530.

Despite these disclosures, improvements in efficacy are highly desirable for compositions of Dabigatran etexilate administrable in the oral cavity. The present invention is directed to pharmaceutical formulations and methods of administration providing enhanced aqueous stability and overcoming both solubility-limited oral absorption and stability limitations of Dabigatran etexilate in order to improve its clinical and therapeutic profile.

### SUMMARY OF THE INVENTION

In a first general aspect, the present invention relates to pharmaceutical compositions for administration of Dabigatran etexilate through the mucous membranes of the oral cavity, comprising a therapeutically effective amount of Dabigatran etexilate, a solubilizer for Dabigatran etexilate and a and a water-soluble film forming agent. The solubilizer is selected from at least one of a water soluble derivative of tocopherol and a polymeric derivative of sorbitan. The solubilizer may be present in the composition in an amount of 1 to 10% (w/w). The pharmaceutical compositions preferably are sold film-shaped compositions for administration in the oral cavity. The water-soluble film forming agent preferably is a water-soluble cellulose

In one aspect of the compositions, the water soluble derivative of tocopherol is a tocopherol polyethylene glycol succinate.

In one aspect, the polymeric derivative of sorbitan is a polyoxoethylene sorbitan monooleate, such as the agents commercialized as Tween or a fatty acid derivative of sorbitan, such as agents commercialized as Span.

In one aspect, the solubilizer can be a combination of a water soluble derivative of tocopherol and a polymeric derivative of sorbitan, wherein the agents are defined as above.

In one aspect, the water soluble derivative of tocopherol is a tocopherol polyethylene glycol succinate.

In one aspect of the invention the pharmaceutical compositions comprise 10 to 60% (w/w) Dabigatran etexilate and 1 to 10% (w/w) of the solubilizer.

In one aspect of the invention the pharmaceutical composition comprise 10 to 60% (w/w) Dabigatran etexilate and 1 to 10% (w/w) of tocopherol polyethylene glycol succinate, as defined above and 1 to 10% (w/w) of a polyoxoethylene sorbitan monooleate, as defined above, for example 1 to 5% (w/w) of tocopherol polyethylene glycol succinate, as defined above and 1 to 5% (w/w) of a polyoxoethylene sorbitan monooleate, as defined above.

In one aspect of the invention, the solid film-shaped pharmaceutical composition having one or several layers, further comprises a co-solvent. The co-solvent comprises at least one polyol. Preferably, the polyol is selected from at least one of polyethylene glycol, glycerol and propylene glycol.

According to one aspect of the invention, the solid film-shaped pharmaceutical composition comprises 60% (w/w) or less of Dabigatran etexilate, 50% (w/w) or less of the water-soluble cellulose, 1 to 10% (w/w) of a tocopherol polyethylene glycol succinate (TPGS) as a solubilizer, and at least one co-solvent selected from polyethylene glycol, glycerol and propylene glycol.

In one embodiment, the solid film-shaped pharmaceutical composition comprises in a single layer 5 to 40% (w/w) of Dabigatran etexilate, 10 to 50% (w/w) of water-soluble cellulose, 1 to 10% (w/w) of a tocopherol polyethylene glycol succinate (TPGS), 1 to 10% (w/w) of a polyoxoethylene sorbitan monooleate; and 1 to 10% (w/w) each of polyethylene glycol, glycerol and propylene glycol.

In one particular embodiment, the solid film-shaped pharmaceutical composition comprises in a single layer about 30 to 40% of (w/w) of Dabigatran etexilate; about 40 to 50% (w/w) of water-soluble cellulose; about 3 to 4% (w/w) of polyethylene glycol; about 4 to 5% (w/w) of glycerol; about 4 to 5% (w/w) of propylene glycol; and about 4 to 5% (w/w) of a tocopherol polyethylene glycol succinate (TPGS).

According to another aspect of the invention, the solid film-shaped pharmaceutical composition comprises a first layer without any solubilizer, comprising 10 to 60% (w/w) of Dabigatran etexilate, 20 to 80% (w/w) of the water-soluble cellulose, and at least one co-solvent selected from polyethylene glycol, glycerol and propylene glycol, a second layer without any Dabigatran etexilate, comprising 20 to 80% (w/w) of the water-soluble cellulose, 1 to 10% (w/w) of a tocopherol polyethylene glycol succinate (TPGS) as a solubilizer, and at least one co-solvent selected from polyethylene glycol, glycerol and propylene glycol.

In one embodiment, the solid film-shaped pharmaceutical composition having two layers, comprises a first layer without any solubilizer, comprises 10 to 60% (w/w) of Dabigatran etexilate, 20 to 80% (w/w) of the water-soluble cellulose, and 1 to 10% (w/w) each of polyethylene glycol, glycerol and propylene glycol, and a second layer without any Dabigatran etexilate, comprising 20 to 80% (w/w) of the water-soluble cellulose, 1 to 10% (w/w) of a tocopherol polyethylene glycol succinate (TPGS), 1 to 10% (w/w) of a polyoxoethylene sorbitan monooleate, and 1 to 10% (w/w) each of polyethylene glycol, glycerol and propylene glycol.

In one particular embodiment, the solid film-shaped pharmaceutical composition having two layers, comprises a first layer without any solubilizer, comprising about 30 to about 40% (w/w) of Dabigatran etexilate, about 40 to about 50% (w/w) of the water-soluble cellulose, about 3 to about 4% (w/w) of polyethylene glycol, about 4 to about 5% (w/w) glycerol, and about 4 to about 5% (w/w) of propylene glycol, and a second layer without any Dabigatran etexilate, comprising about 60 to about 70% (w/w) of the water-soluble cellulose, about 6 to about 8% (w/w) of a tocopherol polyethylene glycol succinate (TPGS), about 5 to about 6% (w/w) of polyethylene glycol, about 7 to about 8% (w/w) glycerol, and about 7 to about 8% (w/w) of propylene glycol. According to another aspect of the invention, the pharmaceutical composition is a solid film-shaped pharmaceutical composition having one or several layers is manufactured from a nanocrystal dispersion of Dabigatran etexilate, comprising 5 to 50% (w/w) of Dabigatran etexilate mixed with a gel, comprising 10 to 70% (w/w) of water-soluble cellulose, 1 to 10% (w/w) of tocopherol polyethylene glycol succinate (TPGS), and at least one co-solvent selected from polyethylene, glycerol and propylene glycol.

In one embodiment of the pharmaceutical composition is a solid film-shaped pharmaceutical composition having one or several layers is manufactured from a nanocrystal dispersion of Dabigatran etexilate, comprising 5 to 50% (w/w) of Dabigatran etexilate mixed with a gel of water soluble cellulose, wherein the gel comprises 10 to 70% (w/w) of water-soluble cellulose, 1 to 10% (w/w) of tocopherol polyethylene glycol succinate (TPGS), 1 to 10% (w/w) of a polyoxoethylene sorbitan monooleate, and 1 to 10% (w/w) each of polyethylene glycol, glycerol and propylene glycol.

In one embodiment of the pharmaceutical composition is a solid film-shaped pharmaceutical composition having one or several layers is manufactured from a nanocrystal dispersion of Dabigatran etexilate, comprising 10 to 30% (w/w) Dabigatran etexilatemixed with a gel of water soluble cellulose, wherein the gel comprises 30 to 70% (w/w) water-soluble cellulose; 1 to 7% (w/w) of tocopherol polyethylene glycol succinate (TPGS); 1 to 5% (w/w) of polyoxoethylene sorbitan monooleate; 1 to 5% (w/w) polyethylene glycol; 1 to 7% (w/w) glycerol; and 1 to 7% (w/w) propylene glycol.

In one particular embodiment of the pharmaceutical composition is a solid film-shaped pharmaceutical composition having one or several layers is manufactured from a nanocrystal dispersion of Dabigatran etexilate, comprising about 15 to about 25% (w/w) Dabigatran etexilatemixed with a gel of water soluble cellulose, wherein the gel comprises about 60 to about 70% (w/w) water-soluble cellulose; about 6.5% (w/w) to about 7.5% (w/w) of tocopherol polyethylene glycol succinate (TPGS); 5 to 6% (w/w) polyethylene glycol; about 6.5 to about 7.5% (w/w) glycerol; and about 6.5 to about 7.5% (w/w) propylene glycol. The water-soluble cellulose described in the pharmaceutical compositions, preferably is hydroxypropyl methylcellulose (HPMC).

The described compositions can further comprise additives, such as buffering agents, sweeteners, flavours and colouring agents.

According to another main aspect of the invention, there is provided a method of manufacturing a solid film or layer comprising Dabigatran etexilate useful in any of the compositions having one or several layers outlined above. The method comprise providing a first solution by dissolving Dabigatran etexilate in solvent comprising acetonitrile and water, providing a second solution comprising the water soluble film or layer forming agent, the solubilizer and optionally at least one co-solvent, admixing first and solution to film-forming solution, and casting the film-forming solution in a film coater and drying it to film product adminsterable to the oral cavity. The film forming agent and the solubilizer are selected as defined above and amounts as defined above. In a preferred embodiment of the method, the solvent for Dabigatran etexilate comprises about 90 to 95% (w/v) of acetonitrile and about 5 to 10% of water.

According to another general aspect of the invention, the pharmaceutical compositions are in the form of a solution, suitable for dropwise administration or for administration as a spray. The solution can further comprise a co-solvent comprising at least one polyol. Suitable polyols are exemplified by propylene glycol and polyethylene glycol. For example, a solution for dropwise administration can comprise tocopherol polyethylene glycol succinate, propylene glycol and polyethylene glycol in order to solubilize and to provide bioavailability of dabigatran etexilate. For example a solution for administration as a spray can comprise tocopherol polyethylene glycol succinate and propylene glycol in order to solubilize and provide bioavailability of dabigatran etexilate.

According to another aspect of the invention, the pharmaceutical composition is in the form of a solid sublingual pharmaceutical composition, the sublingual pharmaceutical composition comprises a water-soluble disintegrating agent.

### BRIEF DESCRIPTION OF THE DRAWINGS

FIG. 1 shows in vitro dissolution of thin film formulations (A, B and C) of Dabigatran etexilate in physiologically relevant buffer media at 37° C.

FIG. 2 shows the in vitro dissolution of film formulations (B and C) of Dabigatran etexilate in human saliva at 37° C.

FIG. 3 shows the simulated pharmacokinetics of Dabigatran etexilate in healthy and moderately renal impaired subjects following sublingual administration of 30 mg of Dabigatran etexilate assuming 50% bioavailability of sublingually administered dose. The pharmacokinetics of 150 mg oral tablets of Dabigatran etexilate is also shown for comparative purposes.

FIG. 4 shows a comparison of the X-ray diffraction patterns of API in oral thin films A, B and C.

### DEFINITIONS

Active therapeutic moiety—a part of medicinal product for use in the treatment of a patient

Adverse event—any undesirable experience associated with the use of a medical product in a patient.

Aqueous solubility—the quality or condition of being soluble in water

Bioavailability—the proportion of a drug or other substance which enters the circulation when introduced into the body.

Bioequivalence—according to the USFDA, it is the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. 21CFR320.1

Cellulose—(acetate), is the acetate ester of cellulose, used as a film coating material for tablets

Clinical doses—doses of a medical product administered to patients

Co-solvent—a secondary solvent added in small quantities to enhance solubility. Co-solvent is usually added to enhance solubility of a solute in the primary solvent. In the present context a co-solvent is used together with a solubilizer.

Dropwise administration—a drop of liquid formulation that is administered to oral mucosa.

Film-forming agents—a group of chemicals that form a pliable, cohesive, and continuous matrix; a water-soluble film forming agent, in this case helps to form a film

First pass effect—also known as first-pass metabolism or presystemic metabolism, is a phenomenon of drug metabolism whereby the concentration of a drug is greatly reduced before it reaches the systemic circulation.

Functional excipient—formulated alongside the active ingredient of a medication included for the purpose of long-term stabilization, bulking up solid formulations that contain potent active ingredient, or to confer a therapeutic enhancement of the active ingredient in the final dosage form, such as facilitating drug absorption, or enhancing solubility.

Intrinsic “passive permeability”—the flux of solutes across a cell membrane by simple diffusion at a rate proportional to the difference in concentration of the solute across the membrane.

Nanosuspension—a sub-micron colloidal dispersions of pure particles of drug, which are stabilized by surface active agents or which are generally used to stabilize nanoparticles against aggregation.

Matrix—a substance in which particles of a medical product are embedded

Membrane permeability—the quality of a cell's plasma membrane that allows substances to pass in and out of it.

Microenvironment—the physical and chemical nature of the medium surrounding a substance of interest

Mucous membrane—an epithelial tissue which secretes mucus, and lines many body cavities and tubular organs including the gut and respiratory passages.

Oral absorption/oral route administration—the most widely employed extravascular route of administration is the oral route. In this case, the drug first reaches the stomach where it usually disintegrates and dissolves in the gastric lumen and is then evacuated in the small intestine, the primary site for absorption.

Oral cavity—the cavity of the mouth; especially the part of the mouth behind the gums and teeth that is bounded above by the hard and soft palates and below by the tongue and by the mucous membrane connecting it with the inner part of the mandible.

Oral film—oral drug strip to administer drugs for absorption in the mouth (buccally or sublingually).

Oral mucosa—any covering membrane starting from the mouth/lips and covering the entire oropharyngeal region of the oral cavity.

Oral route administration—(see oral absorption)

Oropharyngeal mucosa—the mucous membrane lining the inside of the mouth and consists of stratified squamous epithelium termed oral epithelium and an underlying connective tissue termed lamina propria and the mucosa of the nasopharynx, the oropharynx, and the laryngopharynx.

Parenteral administration—intended for administration as an injection or infusion. Common injection types are intravenous (into a vein), subcutaneous (under the skin), and intramuscular (into muscle). Infusions typically are given by intravenous route.

Peroral administration—administration by way of the mouth

Pharmaceutical formulations—may refer to solid, liquid or semi-solid formulations which can enable this method of administration. Some examples of such pharmaceutical formulations used in the field of art include tablets, films, solutions, suspensions, sprays, gels, creams etc. One skilled in the art may be able to prepare different variants of the disclosed compositions to achieve the desired solubility and stability profile. The compositions of the pharmaceutical formulations may be such that the active therapeutic moiety is presented to the oropharyngeal, buccal or sublingual mucosa in a dissolved or rapidly dissolvable state to facilitate transmucosal permeation across the intended site of absorption. Beyond the active DTI moiety and excipients disclosed here, pharmaceutical formulations may present other excipients including but not limited to buffers, pH adjusters, preservatives, solubilizers, stabilizing agents, permeation enhancers, viscosity modifiers, taste masking agents, flavoring agents, antioxidants, solvents, fillers, binders, bulking agents, polymers etc.

Plasma level—amount of a drug/compound in blood plasma

Plasticizer—additive that increase the plasticity or fluidity of a material

Polymeric derivative of sorbitan—excipients such as Tween 80 prepared by pegylation of sorbitan. These are also referred to as polysorbates.

Polyol—a compound (for example a sorbitol or pentaerythritol) containing usually several alcoholic hydroxyl groups; in this case it is used as a sweetener.

Precipitation—the phenomenon of a substance undergoing a phase change from a miscible state to an immiscible state.

Primary solvent—the major solvent constituent in a mixture of solvents.

Prodrug—a biologically inactive compound that can be metabolized in the body to produce a drug.

Residual unabsorbed drug—a quantity of remaining drug that has not been absorbed

Solubility-limited absorption—the absolute amount of drug absorbed is limited by its poor solubility in water.

Solubilizer—an agent that increases the solubility of a substance; in the current case, TPGS is used as a solubilizer for Dabigatran etexilate.

Solubilizing system—a system, by use of which the solubility of materials which were previously insoluble or poorly soluble has been increased in a given media by use of other materials

Steady state concentration—when the rate of drug input is equal to the rate of drug elimination, steady state has been achieved.

Sublingual composition—a composition meant to be placed under a tongue

Sublingual mucosa—the mucosa under the tongue

Surfactant—a substance, such as a detergent, added to a liquid to increase its spreading or wetting properties by reducing its surface tension

Therapeutic dose—the quantity of any substance required to effect the cure of a disease or to correct the manifestations of a deficiency of a particular factor in the diet

TPGS—a water soluble derivative of tocopherol is a polymeric derivative of tocopherol with surface active characteristics. One example of such derivative is a tocopherol polyethylene glycol succinate (TPGS). One specific compound is TPGS 1000.

Trough concentration—the plasma level of a pharmaceutical product measured just before the next dose, Cₘᵢₙ; the opposite of maximal concentration (Cₘₐₓ).

Water soluble derivative of tocopherol—any tocopherol-based excipient formed by chemical modification of tocopherol wherein such a modification renders tocopherol more water soluble than it pure form

Water soluble disintegrating agent—usually a polymer that facilitates disintegration of a solid dosage form. Polyvinylpyrolidone, crocarmellose sodium, sodium starch glycolate are examples of disintegrants.

### DETAILED DESCRIPTION OF THE INVENTION

Table I shows the aqueous solubility of Dabigatran in presence of various pharmaceutically relevant solubility enhancers under physiological pH conditions relevant to oral absorption (pH 4.5-7.4). Table II shows the percent of Dabigatran etexilate remaining after exposing Dabigatran etexilate to different aqueous solutions for 15 minutes at 20-40° C.

TABLE I

| | | Solubility (mg/ml) |
|---|---|---|
| Composition | pH | 25° C. |
| Water | 6.5 | <0.001 |
| 10% w/v PEG 400 in 0.01M | 7.5 | <0.1 |
| histidine buffer | | |
| 2% w/v Polysorbate 80 in | 7.4 | <0.1 |
| 0.01M histidine buffer | | |
| 5% w/v Polysorbate 80 in | 7.5 | <0.1 |
| 0.01M histidine buffer | | |
| 10% w/v Glycerol in 0.01M | 7.6 | <0.1 |
| histidine buffer | | |
| 5% w/v TPGS in 0.01M | 7.6 | 0.37 |
| histidine buffer | | |

TABLE II

| | % of Initial | % of Initial |
|---|---|---|
| Solution Composition | (23° C.) | (40° C.) |
| Water | 21.6 | 21.0 |
| pH 2 acetate buffer (0.1M) | 0.2 | 0.2 |
| pH 3 acetate buffer (0.1M) | 22.4 | 22.2 |
| pH 4 phosphate buffer (0.1M) | 59.5 | 55.7 |

However, based on data presented in Table I, TPGS (tocopherol polyethylene glycol succinate) would be expected to be a poor solubilizer by one skilled in the art. Even with use of 5% TPGS which is significantly greater that level typically used in pharmaceutical formulations, solubility of Dabigatran Etexilate could only be raised to 0.37 mg/ml. The solubility data demonstrate that Vitamin E TPGS is a very weak solubilizer at best.

In addition, the data disclosed in Table II suggest that Dabigatran is highly susceptible to degradation.

The present invention provides pharmaceutical compositions and method of administration of Dabigatran etexilate to overcome the clinical limitations posed by gastrointestinal delivery, specifically by increasing the solubility of Dabigatran etexilate, while maintaining it in its most bioavailable form to ascertain an effective transport through the oral mucous membranes so it effectively can exert its anticoagulant activity. We have observed that the use of pharmaceutical solubilizers such as d-alpha Tocopherol polyethylene glycol succinate (TPGS) and Polysorbate 80 improved the solubility up to 5-10 mg/mL. Without being bound to any specific mechanism, the enhanced solubility and dissolution of Dabigatran etexilate, when co-formulated with TPGS may likely be due to ability of TPGS to inhibit precipitation and maintain supersaturation of dabigatran free base in the formulation microenvironment during dissolution. Pharmaceutical co-solvents such as Polyethylene Glycol 400 (PEG 400) and Glycerol improved the solubility compared to water alone, but the solubility was still<0.1 mg/mL.

### Methods

### Preparation of Films

Three different types of films were prepared by the solvent casting process. Tables III through V disclose useful pharmaceutical compositions according to the invention with enhanced solubility and stability in aqueous solutions.

Table III discloses useful composition of an oral film of Dabigatran etexilate (Formulation A, Oral Film F1).

TABLE III

| | | Composition | Composition |
|---|---|---|---|
| | | (% w/w) | (% w/w) |
| Ingredients | Function | (Preferable) | (More Preferable) |
| Dabigatran | API | <60 | 5-40 |
| HPMC | Film former | <50 | 10-50 |
| PEG 400 | Plasticizer | 1-10 | 1-5 |
| Glycerol | Plasticizer | 1-10 | 1-5 |
| Propylene glycol | Plasticizer | 1-10 | 1-5 |
| T80 | Surfactant | 1-10 | 1-5 |
| TPGS 1000 | Solubiliser | 1-10 | 1-5 |
| Acesulfame | Sweetner | 1-10 | 1-5 |
| Strawberry | Flavour | 1-10 | 1-5 |
| Colour blue | Colour | 1-10 | 1-5 |

Table IV discloses the composition of a multilayered oral film of Dabigatran etexilate (Formulation B, Oral Film F2).

TABLE IV

| | | Non-drug layer % | | Drug layer % | |
|---|---|---|---|---|---|
| | | (w/w) | | (w/w) | |
| | | Prefer- | More | Prefer- | More |
| Ingredients | Function | able | Preferable | able | preferable |
| Dabigatran | API | — | — | 10-60 | 37.2 |
| HPMC | Film former | 20-80 | 40-70 | 20-80 | 40-70 |
| PEG 400 | Plasticizer | 1-10 | 2-6 | 1-10 | 2-6 |
| Glycerol | Plasticizer | 1-10 | 1-5 | 1-10 | 1-5 |
| Propylene | Plasticizer | 1-10 | 1-5 | 1-10 | 1-5 |
| glycol | | | | | |
| T80 | Surfactant | 1-10 | 1-5 | 1-10 | 1-5 |
| TPGS 1000 | Solubiliser | 1-10 | 1-5 | — | — |
| Acesulfame | Sweetner | 1-10 | 1-5 | — | — |
| Strawberry | Flavour | 1-10 | 1-5 | 1-10 | 1-5 |
| Colour blue | Colour | 1-10 | 1-5 | 1-10 | 1-5 |

Table V discloses the composition of an oral film of comprising dispersed Dabigatran Etexilate nanocrystals (Formulation C, Oral Film F3).

TABLE V

| | | Nanocrystal dispersion | | Other excipients (gel) | |
|---|---|---|---|---|---|
| | | 5%(w/w) | | %(w/w) | |
| | | | More | | More |
| Ingredients | Function | Preferable | Preferable | Preferable | Preferable |
| Dabigatran^{†} | API | 5-50 | 10-30 | — | — |
| HPMC | Film former | — | — | 10-70 | 30-70 |
| PEG 400 | plasticizer | — | — | 1-10 | 1-5 |
| Glycerol | Plasticizer | — | — | 1-10 | 1-7 |
| Propylene glycol | Plasticizer | — | — | 1-10 | 1-7 |
| T80 | Surfactant | — | — | 1-10 | 1-5 |
| TPGS 1000 | Solubiliser | — | — | 1-10 | 1-7 |
| Acesulfame | Sweetner | — | — | 0.2-5 | 0.5-5 |
| Strawberry | Flavour | — | — | 0.1-5 | 0.5-3 |
| Colour blue | Colour | — | — | 0.001-1 | 0.005-0.5 |

Dabigatran (DAB) and different functional excipients (For F1 and F2) were premixed in water/ACN and magnetically stirred at 500 rpm for 3 hours at 30-40° C. In case of F3, DAB nanosuspension prepared separately (see 1.2) and then mixed with polymer casting mass. The resulting casting film mass of all batches were kept aside to remove air bubbles. Films of all batches were casted on a release liner known in the art, for example SCOTCH PACK 3M Inc, USA using an automated film applicator equipped with a coating knife (Erichsen, Sweden). The fixed wet mass thickness (550 microns) and casting speed (5 mm/sec) were used for casting of films. The cast films were dried for 30 min at 60° C. and stored in desiccator (23° C./30% RH).

### Nanosuspension Preparation

DAB (0.8 g) was added to 0.1% w/v Polysorbate 80 aqueous water (3.2 g). The suspension was subjected to high shear homogenization (T 25 ULTRA-TURRAX Disperser, IKA) at 8-10° C. for 15 min at 16000 rpm.

### Thickness

Digital micrometer (0.001 mm, Mitutoyo, Japan) was used to measure thickness of the alginate-based films. Average values of ten measurements in different regions of each sample were calculated and used in the calculation of water vapor permeability and tensile properties

### Transparency

Ultraviolet-visible (UV) spectroscopy (Shimadzu, UV 1650PC, UV-visible spectrophotometer) was used to determine the transparency of the films. The oral films were located in the spectrophotometer cell after that they had been cut into rectangle pieces. The air was a reference. The absorption values were measured at 500 nm. By using the following equation the opacity of the film was calculated:

Opacity measurement=A₅₀₀500/T

A₅₀₀ is the absorption value which was measured at 500 nm and T is the thickness of the film in microns. The average and standard deviations were calculated.

### Folding Endurance

The number of times a film can be folded without breaking or visibly cracking is defined as the folding endurance. Liew et al.'s method was used to determine the folding endurance. The sample films (3λ3 cm2) ends was held with forceps and folded to 180° direction till break or limit to 100 times.

### Differential Scanning Calorimetry (DSC) and Modulate DSC (MDSC)

Thermal analysis of the samples was performed using conventional and modulated differential scanning calorimeter (TA Instruments Q 1000, USA). DSC is equipped with refrigerated cooling system and was previously calibrated for melting enthalpy and heat capacity using indium and sapphire respectively. Each sample (2-3 mg) was placed in an aluminum pan and hermetically sealed with an aluminum lid. Nitrogen was purged at 50 ml/min in all experiments. DSC experiments were performed at 10° C./min heating rate up to 300° C. Experimental parameters in MDSC were; temperature range −10° C. to 225° C., heating rate 2° C./min, modulation amplitude 0.747° C. and period 60 sec. The data were acquired using Thermal Advantage software and analyzed by Universal Analysis software (TA Instruments).

### Thermogravimetric Analysis TGA

The samples were filled in platinum TGA sample pans. All measurements were performed at 10° C./min heating rate up to 220° C. and nitrogen was purged at 50 ml/min.

### Powder X-Ray Diffraction PXRD

PXRD patterns of film samples were obtained by X-ray Diffractometer (X'Pert PRO, PANalytical, Sweden) with Ni-filtered Cu—K radiation in the 2θ (diffraction angle) between 5° and 40° at 45 kV voltage and current of 40 mA radiation. All of the patterns were obtained at 25±1° C.

### Dynamic Vapor Sorption Analysis

The studies were performed using a Dynamic Vapour Sorption apparatus (DVS, Surface Measurement Systems, London, UK) to determine the stability of the product to moisture. The apparatus consists of a Cahn microbalance housed inside a temperature-controlled cabinet. All experiments were performed at 25° C. Dry nitrogen was bubbled through water to give 100% relative pressure of the solvent. The relative pressure of water flowing past the sample is controlled via a computer program which sets the appropriate flow to the wet (100% relative pressure water) and dry side (dry nitrogen).

### Mechanical Properties

Mechanical testing was conducted on a TA.XTPlus texture analyzer (Stable Micro Systems, Godalming, UK) equipped with a 5 Kg load cell as previously described. Briefly, samples of casted films were cut in rectangular strips of 1×5 cm2 and 1 cm on each end were held between clamps attached to the texture analyzer, thus the effective testing area was 1×3 cm2. The upper clamp (connected to the mobile arm of the texture analyzer) stretches the film upwards at a rate of 0.5 mm/sec until film rupture. Stress is obtained from the force measurements obtained from the instrument divided by the cross-sectional area of the film, while strain is computed by dividing the increase in length by the initial film length. From the stress vs. strain plot, the tensile strength (TS) and the elongation at break (EB) are obtained from the peak stress and the maximum strain, respectively, also represented by the following equations:

$$Tensile strength ( TS ) = \frac{Peak stress}{Cross \text{-} sectional area of film}$$

$$Elongation at break ( EB ) = \frac{Increase in length at break}{Initial film length} \times 100$$

Additionally, the elastic modulus (EM) was obtained from the initial elastic deformation region in the stress vs. strain plot. Since the rate of the mobile arm extension was constant for all samples tested, direct comparison of the slope in this region can be done.

### Particle Size

The nanosuspension of DAB freshly prepared (1 in 10 ml dilution) were filled in disposable polystyrene cells for particle size measurement by dynamic laser light scattering technique using Zetasizer (Malveran, UK). The particle size was determined from triplicate measurement of 50 accumulation time of each sample. The film samples (1×1 cm²) were dissolved in 10 ml of 1% tween 80 solutions and followed above particle size analysis.

### Drug Content

The drug loaded film (1×1 cm) was dissolved in 15 ml of ACN:water (70:30) and the filtered solution was quantified by HPLC. Duplicates were measured per formulation. Edge pieces with deviating thickness were excluded.

### Disintegration

One piece of film (1×1 cm) was placed into a petri dish. After adding two milliliters of water, the petri dish was shaken constantly at 80 rpm. Time until the film fully rupture was measured.

### Dissolution

Cast films (equivalent to 40 mg of F1, F2 and 20 mg of F3) were used for in-vitro dissolution studies in saliva (non-sink) and aqueous media (sink) conditions. 10 ml of freshly collected human saliva and 900 ml of Millipore water with 1% Tween 80 solution was used as a dissolution medium in non-sink and sink conditions, respectively. Media were stirred at 80 and 100 rpm at 37±0.5° C. in respective studies. The samples (0.5 ml in non sink and 5 ml in sink conditions) were withdrawn at preset time intervals and simultaneously replaced with similar amount of respective dissolution media. The samples were filtered using 0.45 micron syringe filters and the filtrate was analyzed by HPLC method.

Pharmaceutical compositions and processes for preparing these compositions disclosed here were surprisingly able to achieve stable aqueous solutions of Dabigatran etexilate that can also overcome the solubility limitation under physiological conditions. The disclosed formulations used TPGS as a solubility enhancer but the solubility and stability improvement observed from these formulations was substantially greater than that anticipated from the solubility and stability experiments depicted in Tables I & II. Table VI shows the amount of Dabigatran etexilate dissolved in aqueous media 10 mL of water (pH>4) or saliva (pH 6.5) at 37° C.

TABLE VI

| | | TPGS in | | Cumulative amount | | Cumulative amount | |
|---|---|---|---|---|---|---|---|
| | | dissolution | | (mg) dissolved in | | (mg) dissolved in | |
| | Dose | medium | | Water^{†} | | human saliva^{‡} | |
| | (mg) | mg | % w/v | Mean (SD) | | Mean (SD) | |
| Formulation A | 40 | 5.27 | 0.05 | 29.4 | (3.5) | 33.8 | (6.8) |
| Formulation B | 40 | 3.76 | 0.38 | 33.5 | (5.8) | 29.6 | (4.1) |
| Formulation C | 20 | 2.72 | 0.27 | 11.1 | (0.7) | 9.8 | (3.4) |

Table VII discloses the composition of an oral film of Dabigatran etexilate (Formulation A).

TABLE VII

| | | Composition |
|---|---|---|
| Ingredients | Function | (% w/w) |
| Dabigatran | API | 35.2 |
| HPMC | Film former | 44.0 |
| PEG 400 | Plasticizer | 3.3 |
| Glycerol | Plasticizer | 4.3 |
| Propylene glycol | Plasticizer | 4.4 |
| T80 | Surfactant | 2.1 |
| TPGS 1000 | Solubiliser | 4.4 |
| Acesulfame | Sweetner | 1.1 |
| Strawberry | Flavour | 1.1 |
| Colour blue | Colour | 0.01 |

Table VIII discloses the composition of a multilayered oral film of Dabigatran etexilate (Formulation B).

TABLE VIII

| | | Non-drug layer | | Drug layer | |
|---|---|---|---|---|---|
| | | Weights of | Weights of | Weights of | Weights of |
| | | materials | materials | materials | materials |
| Ingredients | Function | in grams^{†} | in % w/w | in grams^{†} | in % w/w |
| Dabigatran | API | — | — | 16 | 37.2 |
| HPMC | Film former | 1.5 | 66.7 | 20 | 46.5 |
| PEG 400 | Plasticizer | 2 | 5.3 | 1.5 | 3.5 |
| Glycerol | Plasticizer | 2 | 7.1 | 2 | 4.7 |
| Propylene glycol | Plasticizer | 1 | 7.1 | 2 | 4.7 |
| T80 | Surfactant | 2 | 3.5 | 1 | 2.3 |
| TPGS 1000 | Solubiliser | 0.5 | 7.0 | — | — |
| Acesulfame | Sweetner | 0.49 | 1.8 | — | — |
| Strawberry | Flavour | 0.01 | 1.7 | 0.49 | 1.1 |
| Colour blue | Colour | 19 | 0.03 | 0.01 | 0.02 |

^{†}based on 100 g of film weight

Table IX discloses the composition of an oral film of comprising dispersed Dabigatran etexilate nanocrystals (Formulation C).

TABLE IX

| | | Nanocrystal disperson | | Other excipients (gel) | |
|---|---|---|---|---|---|
| | | Weights of | Weights of | Weights of | Weights of |
| | | materials | materials | materials | materials |
| Ingredients | Function | in grams | in % w/w | in grams | in % w/w |
| Dabigatran^{†} | API | 0.8 | 20% | — | — |
| HPMC | Film former | — | — | 19 | 66.7 |
| PEG 400 | plasticizer | — | — | 1.5 | 5.3 |
| Glycerol | Plasticizer | — | — | 2 | 7.0 |
| Propylene glycol | Plasticizer | — | — | 2 | 7.0 |
| T80 | Surfactant | — | — | 1 | 3.5 |
| TPGS 1000 | Solubiliser | — | — | 2 | 7.0 |
| Acesulfame | Sweetner | — | — | 0.5 | 1.8 |
| Strawberry | Flavour | — | — | 0.49 | 1.7 |
| Colour blue | Colour | — | — | 0.01 | 0.0 |

^{†}Nanocrystal dispersion is mixed with gel in 40:60 ratio before film casting.

Under physiological conditions relevant to oral absorption (pH 4.5-7.4), the use of 5% w/v TPGS resulted in solubility improvement of Dabigatran etexilate up to 0.4 mg/mL in water. The formulations disclosed in Tables VII through IX employed TPGS in the range of 0.05-0.4% w/v which is an order of less than that used in solubility studies. However the disclosed formulations were able to achieve solution concentrations up to 3 mg/mL. While the mechanism for this enhanced solubility demonstrated by the proposed formulations is not known, the resulting solution concentrations may be supersaturated. In addition, Dabigatran etexilate was observed to be chemically stable during the course of the dissolution studies conducted with the proposed formulations. By overcoming the solubility and stability limitations, the disclosed pharmaceutical formulations may substantially improve the pharmacokinetics Dabigatran etexilate. Due to the improved stability, solubility and consequently the potential for greater absorption, it is possible to achieve the same therapeutic levels of Dabigatran by use of much lower dose of the active.

The method of administration proposed in this disclosure also allows for substantial mitigation of exposure of the active to the gastrointestinal tract since the target site of delivery is the opening of the oral cavity, buccal and sublingual mucosa. The rapid dissolution of pharmaceutical formulations that enable this method of administration allows for fast in situ absorption, thereby minimizing undissolved active substance in the buccal, sublingual oropharyngeal region. Thus, this method also minimizes excess residual drug in any part of the gastrointestinal tract. Therefore the risk of local side-effects such as abdominal pain, gastric hemorrhage, gastrointestinal ulcers can be substantially reduced relative to the current methods of administration. Since gastrointestinal side-effects are a limiting factor for better patient compliance and a more wide-spread usage of the therapeutic moiety, the proposed method of administration allows for improved patient compliance.

The maximum plasma concentration, Cₘₐₓ obtained by using this method and pharmaceutical formulations that enable the method of administration may be, but is not limited to 10-150% of the Cₘₐₓ obtained using current clinical reference formulation, Pradaxa®. In one aspect, the Cₘₐₓ obtained by this method of administration may meet bioequivalence standards, as determined by the United States Food and Drug Administration (US FDA), including that the 90% confidence interval of the ratio of the Cₘₐₓ between the test and reference formulation fall within 80-125%. The term “Reference formulation” may refer to formulations of Dabigatran etexilate that are approved for use by the US FDA and through their history of use define the intended therapeutic regimen for effective pharmacotherapy with Dabigatran etexilate.

The area under the plasma concentration-time profile (AUC) by using this method and pharmaceutical formulations that enable the method of administration may be, but is not limited to 10-150% of the AUC obtained using current clinical reference formulation. In one aspect, the AUC obtained by this method of administration may meet bioequivalence standards, as determined by the United States Food and Drug Administration (US FDA), including that the 90% confidence interval of the ratio of the AUC between the test and reference formulation fall within 80-125%.

The time to maximum plasma concentration, Tₘₐₓ obtained by using this method and pharmaceutical formulations that enable the method of administration may be, but is not limited to 10-150% of the Tₘₐₓ obtained using current clinical reference formulation. In one aspect, the Tₘₐₓ obtained by this method of administration may meet bioequivalence standards, as determined by the United States Food and Drug Administration (US FDA), including that the 90% confidence interval of the ratio of the Tₘₐₓ between the test and reference formulation fall within 80-125%.

The following examples will describe the improved method of administration and disclosed pharmaceutical formulations that enable the method of administration of Dabigatran etexilate that constitute the present invention:

### Example 1—Formulation A

0.4 gm of Dabigatran Etexilate and 0.001 g of blue food coloring agent is dissolved in 5.1 gm of Acetonitrile in a screw capped glass vial and the resulting solution is referred to as solution A. In a glass beaker 1.7 gm of Hydroxypropyl Methylcellulose, 0.2 gm of polyethylene glycol 4000, 0.2 gm of glycerol, 0.2 gm of propylene glycol, 0.1 gm of saccharin, 0.049 gm of peppermint flavor were mixed in 2 gm of acetic acid and the resulting solution is referred to as Solution B. Solution A is mixed with Solution B at the rate of 500 RPM at 40° C. The resulting solution was caste at the rate of 6 mm/sec in a film coater and dried over night at room temperature. The resulting film, referred to as Formulation A was cut into desired sizes and packaged in aluminum pouches.

Morphological properties of the film such as appearance, transparency, pealability and flexibility were monitored after storage at controlled room temperature. The mechanical properties of the film such as tensile strength, percent elongation at break and elastic modulus were measured by texture profile analyzer. The physical state of the API in the film was assessed by using powder X-ray diffraction (PXRD), differential scanning calorimetry and microscopy. The time to disintegration of the film was measured by adding a 1×1 cm size film formulation, Formulation A into a petri dish containing 2 mL of a pH 6.8 phosphate buffer and noting the time for rupture of the film.

The In vitro dissolution of the film was monitored by placing film formulation (equivalent to 40 mg of Dabigatran Etexilate) into a beaker containing pH 6.8 phosphate buffer shaken at 50 RPM and maintained at 37° C. Samples were withdrawn from the beaker at 1, 3, 5, 10, 20, 30 and 60 minutes; and analyzed by HPLC for drug content released from the film. The representative In vivo dissolution of the film in the sublingual, buccal or oropharyngeal cavity was studied by observing the dissolution of the film In vitro in human saliva at 37° C. For these studies, the film formulation was placed into 10 mL of human saliva was collected in a beaker, shaken gently at 50 RPM and maintained at 37° C. Samples of saliva were withdrawn from the beaker at 1, 3, 5, 10, 20, 30 and 60 minutes; and analyzed by HPLC for drug content released from the film.

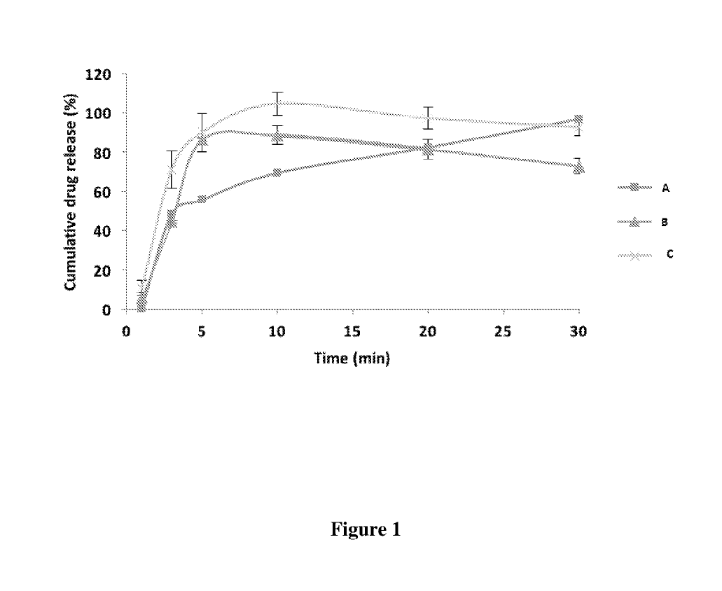

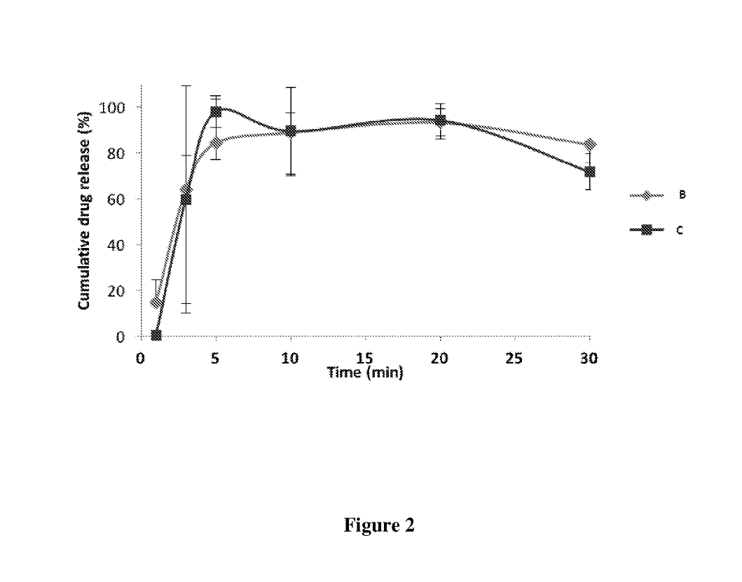

FIG. 1 shows the In vitro dissolution of the film in pH 6.8 phosphate buffer at 37° C. FIG. 2 shows the representative In vivo dissolution of the film in human saliva at 37° C. As can be seen from the dissolution data in FIGS. 1 & 2, >80% of 40 mg of Dabigatran Etexilate in the film formulations intended for the target method of administration can be dissolved in 10 mL of pH 6.8 buffer representative of physiological media or directly in human saliva to achieve concentrations>3 mg/mL which is orders of magnitude greater than the reported solubility of Dabigatran Etexilate at physiological pH conditions in the gastrointestinal tract (pH 4.5-7.4).

### Example 2 Variation of Formulation A

0.4 gm of Dabigatran Etexilate and 0.001 g of blue food coloring agent is dissolved in 5.1 gm of Acetonitrile in a screw capped glass vial and the resulting solution is referred to as solution A. In a glass beaker 1.6 gm of Hydroxypropyl Methylcellulose, 0.2 gm of polyethylene glycol 4000, 0.2 gm of D-α-Tocopheryl polyethylene glycol succinate, 0.2 gm of glycerol, 0.2 gm of propylene glycol, 0.1 gm of saccharin, 0.049 gm of peppermint flavor were mixed in 2 gm of acetic acid and the resulting solution is referred to as Solution B. Solution A is mixed with Solution B at the rate of 500 RPM at 40° C. The resulting solution was caste at the rate of 6 mm/sec in a film coater and dried over night at room temperature. The resulting film, referred to as F-2 was cut into desired sizes and packaged in aluminum pouches as a unit dosage form.

The In vitro dissolution of the film was monitored by placing F-17 film formulation (equivalent to 40 mg of Dabigatran Etexilate) into a beaker containing pH 6.8 phosphate buffer shaken at 50 RPM and maintained at 37° C. Samples were withdrawn from the beaker at 1, 3, 5, 10, 20, 30 and 60 minutes; and analyzed by HPLC for drug content released from the film. The representative In vivo dissolution of the film in the sublingual, buccal or oropharyngeal cavity was studied by observing the dissolution of the film In vitro in human saliva at 37° C. For these studies, the film formulation was placed into 10 mL of human saliva was collected in a beaker, shaken gently at 50 RPM and maintained at 37° C. Samples of saliva were withdrawn from the beaker at 1, 3, 5, 10, 20, 30 and 60 minutes; and analyzed by HPLC for drug content released from the film. FIG. 1 shows the In vitro dissolution of the film in pH 6.8 phosphate buffer at 37° C. FIG. 2 shows the representative In vivo dissolution of the film in human saliva at 37° C.

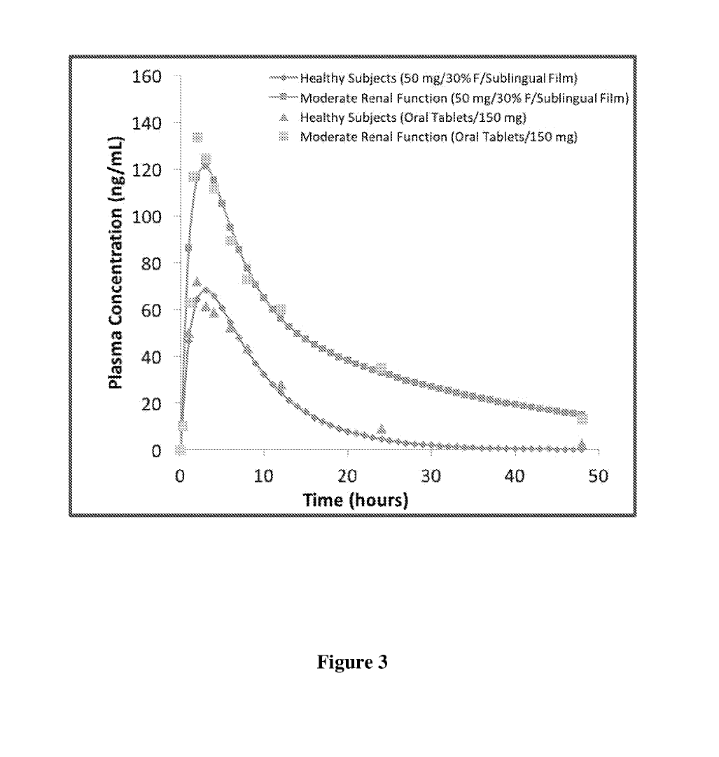

Based on the rapid dissolution and >80% release profile in FIGS. 1 and 2, the pharmacokinetics of film formulations of Dabigatran Etexilate containing 30 mg of active ingredient placed in sublingual mucosa were simulated. In spite of Dabigatran Etexilate being known as a BCS class II compound with high permeability and >80% release of Dabigatran Etexilate being from the formulations in human saliva, only 50% of the administered dose was assumed to be bioavailable as a worst-case scenario. The pharmacokinetic simulations were conducted both for the case of healthy volunteers and patients with moderate renal function; and shown in FIG. 3. The clinically observed pharmacokinetics of Dabigatran Etexilate after administrating 150 mg of the active ingredient in the form of oral tablets is also shown for comparison. As can be seen from FIG. 3, due to the substantially greater solubility afforded by the film formulations intended for sublingual administration of Dabigatran Etexilate, 30 mg of Dabigatran Etexilate is sufficient to match the pharmacokinetic profile of the currently marketed 150 mg Dabigatran Etexilate tablet reference formulation.

### Example 3

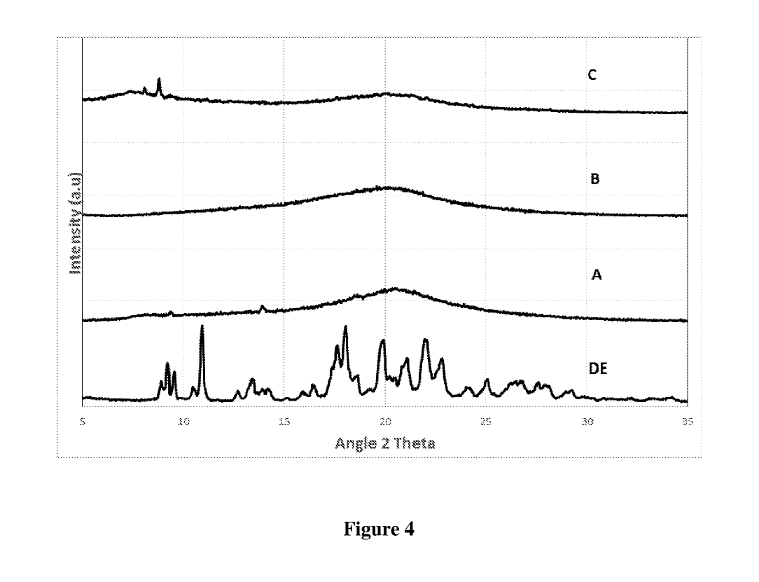

0.4 gm of Dabigatran Etexilate and 1 mg of blue food coloring agent is dissolved in 6.4 gm of Acetonitrile in a screw capped glass vial and the resulting solution is referred to as solution A. In a separate glass beaker 0.8 gm of Hydroxypropyl Methylcellulose, 0.1 gm of glycerol, 0.3 gm of isomalt, 40 mg of peppermint flavor were mixed in 2 gm of acetic acid and the resulting solution is referred to as Solution B. Solution A is mixed with Solution B at the rate of 500 RPM at 40° C. The resulting solution was caste at the rate of 6 mm/sec in a film coater and dried over night at room temperature. The resulting film, referred to as F1 was cut into desired sizes and packaged in aluminum pouches. A 2 cm×1 cm film containing 32 mg of Dabigatran Etexilate was placed in the sublingual or buccal cavity of patients suffering from Atrial fibrillation. The film was held in the sublingual cavity for a period not less than 5 minutes to facilitate absorption of the active ingredient through the sublingual mucosa. The In vitro dissolution of the film, F1 was monitored by placing the film into a beaker containing pH 6.8 phosphate buffer shaken at 50 RPM and maintained at 37° C. Samples were withdrawn from the beaker at 1, 3, 5, 10, 20, 30 and 60 minutes; and analyzed by HPLC for drug content released from the film. The In vitro dissolution is shown in FIG. 4.

### Example 4 Formulation B

1.6 gm of Dabigatran Etexilate and 1 mg of blue food coloring agent is dissolved in 2 gm of Acetonitrile in a screw capped glass vial and the resulting solution is referred to as solution A. In a separate glass beaker 2 gm of Hydroxypropyl Methylcellulose, 0.15 gm polyethylene glycol, 0.1 gm tween 80, 0.2 gm of glycerol, 0.2 gm of propylene glycol, 49 mg of straw berry flavor were mixed in 2 gm of Acetonitrile and 1.7 g of water and the resulting solution is referred to as Solution B. Solution A is mixed with Solution B at the rate of 500 RPM at 40° C. The resulting solution was caste at the rate of 5 mm/sec in a film coater with wet film thickness of ca 500 micrometers and dried at 60° C. for 3 hours in a laboratory oven. The resulting film is a drug-layered thin film, referred to as layer I.

In another glass beaker 1.9 gm of Hydroxypropyl Methylcellulose, 0.15 gm polyethylene glycol, 0.1 gm tween 80, 0.2 gm of Vitamin E TPGS (d-alpha tocopheryl polyethylene glycol 1000 succinate), 0.2 gm of glycerol, 0.2 gm of propylene glycol, 0.05 gm of acesulfame, 49 mg of straw berry flavor and 1 mg of blue food coloring agent were mixed in 7.15 gm of water at the rate of 500 RPM at 40° C. The resulting solution was caste at the rate of 5 mm/sec in a film coater with wet film thickness of 500 micrometers and dried at 60° C. for 3 hours in a laboratory oven. The resulting film is a solubility enhancing excipient layer referred to as layer II Layer I and layer II were cut into desired sizes and sandwiched, referred to as F2 (bi-layer film), and packaged in aluminum pouches. A 2 cm×3 cm film containing 40 mg of Dabigatran Etexilate was placed in the sublingual or buccal cavity of patients suffering from Atrial fibrillation. The film was held in the sublingual cavity for a period not less than 5 minutes to facilitate absorption of the active ingredient through the sublingual mucosa.

### Example 5—Nanodispersion

0.8 gm of Dabigatran Etexilate was added to 3.2 gm of water in a 10 ml glass vial. The resulting suspension is subjected to high shear homogenization at 1600 RPM and 4-5° C. The size distribution of the drug particles was below 500 nm as confirmed using Zeta sizer (Malvern) and this nano-suspension was referred to as solution A. In a separate glass beaker 1.9 gm of Hydroxypropyl Methylcellulose, 0.15 gm polyethylene glycol, 0.1 gm tween 80, 0.2 gm of Vitamin E TPGS (d-alpha tocopheryl polyethylene glycol 1000 succinate), 0.2 gm of glycerol, 0.2 gm of propylene glycol, 0.05 gm of acesulfame, 49 mg of straw berry flavor and 1 mg of blue food coloring agent were mixed in 7.15 gm of water at the rate of 500 RPM at 40° C. The resulting polymer gel was referred to as solution B. Solutions A and B were geometrically mixed using overhead stirrer at ambient temperature (22° C.) and kept outside for the removal of air bubbles. The resulting solution was casted at the rate of 5 mm/sec in a film coater with wet film thickness of ca 500 micrometers and dried at 60° C. for 3 hours in a laboratory oven. This film formulation, referred to as formulation F3 was packaged in aluminum pouches. Table VIII shows the particle size of the API in Formulation C after homogenization and after film formation.

TABLE VIII

| | Particle Size |
|---|---|
| Formulation | (nm) |
| Pre-film (Nanosuspension) | 674 ± 34 |
| Film | 987 ± 103 |

Table IX shows the different physical properties of the Formulation A, B, and C films, as graded on a 10 point scale: 8-10/10 (Good) 5-7/10 (Moderate) and less than 5/10 (Poor).

TABLE X

| Properties | Formulation A | Formulation B | Formulation C |
|---|---|---|---|
| Appearance | G | G | G |
| Peelabllity | G | G | G |
| Touch sense | G | G | G |
| Thickness (microns) | 80 | 80 | 80 |
| Transparency | G | G | M |
| Flexibility | G | G | G |

FIG. 4 shows the X-ray diffraction patterns of the formulations F1, F2 and F3. While the API in formulation F3 exists in a nanocrystalline state, the API is molecularly dispersed in formulations F1 and F2. All three formulations exhibited a rapid disintegration and drug release as depicted in Table XI, which shows the in vitro disintegration time (mins) of oral films of Dabigatran Etexilate for the three formulations.

TABLE XI

| | Drug Loading | Disintegration time |
|---|---|---|
| Formulation | (mg/cm²) | (mins) |
| A | 5.7 ± 0.1 | 4.3 ± 0.4 |
| B | 6.4 ± 0.6 | 4.6 ± 0.3 |
| C | 3.1 ± 0.1 | 0.7 ± 0.3 |

Based on data presented in Table I above, Vitamin E TPGS would be expected to be a poor solubilizer. Even with the use of 5% Vitamin E TPGS, which is significantly greater that level typically used in pharmaceutical formulations, solubility of Dabigatran etexilate could only be raised to 0.37 mg/ml. For this reason, it is surprising that pharmaceutical formulations of the present invention containing<0.1% of Vitamin E TPGS in the dissolution media, which is 50 fold lower than the concentration used in the solubility studies in Table I, result in a solubility in dissolution media corresponding to 3 mg/mL which is 10 fold higher than that obtained with a 50 fold higher Vitamin E

The superior performance may be related to the combination of excipient components and the process used to facilitate intimate mixing of the API and excipients during the film formation process.

The data disclosed in Table II in our application suggest that Dabigatran is highly susceptible to degradation. The use of the excipients disclosed in this application can render the Dabigatran dispersed in the film in an amophorous state rendering it susceptible to degradation. Thus it is important to maintain the drug in a crystalline state while still maintaining the superior dissolution profile. The bilayered films disclosed herein are intended to keep the drug in a crystalline state which still maintaining the superior dissolution profile. The data as presented demonstrate that the present invention successfully attains a bilayer film, able to maintain the drug in a nano-crystalline state, while still able to maintain the dissolution profile. Accordingly, embodiments of the present invention admits to separate incompatible excipients from the drug layer as described.

### Example 6—Drops

6 gm of Dabigatran Etexilate, 10 mg peppermint oil, 5 gms of TPGS, 0.2 gm of saccharin was dissolved in a mixture of 50 mL of Propylene glycol and 50 mL of Polyethylene glycol 400. The formulation was packaged in 5 mL dropper bottles and stored at 2-8° C. in a refrigerator until administration. 0.5 mL of the formulation was withdrawn using a dropper and administered sublingually to a patients at risk of stroke from DVT. The solution was held in the sublingual cavity for a period not less than 5 minutes in the sublingual cavity to facilitate the absorption of Dabigatran Etexilate. Following absorption, the formulation was gulped down throat with the aid of a glass of water.

### Example 7—Spray

3 gm of Dabigatran Etexilate, 1 mg peppermint oil, 2 gms of Vitamin E TPGS, 50 mg of sucralose was dissolved in a mixture of 8 mL of Alcohol, USP and 2 mL of Propylene glycol. The formulation was packaged in a HDPE dispenser spray bottle with a mechanical spray pump such that each spray would yield 0.05 mL of the formulation containing 15 mg of Dabigatran Etexilate. The formulation was administered to the buccal and oropharyngeal region of patients at risk of stroke from Atrial fibrillation by priming the pump, pointing the spray nozzle to the opening of the mouth and pressing the dispenser twice to administer two sprays at the target mucosal sit

1. A solid film-shaped pharmaceutical composition having one or several layers for administration of Dabigatran etexilate through the mucous membranes of the oral cavity, comprising:
5 to 50% (w/w) of Dabigatran etexilate,
a solubilizer for Dabigatran etixilate selected from at least one of a water soluble derivative of tocopherol and a polymeric derivative of sorbitan,
a polyol co-solvent selected from at least one of polyethylene glycol, glycerol and propylene glycol;
and 10-70% (w/w) of a water-soluble film forming agent comprising a water-soluble cellulose,
wherein, said composition is manufactured from a nanocrystal dispersion of Dabigatran mixed with a gel of the water-soluble cellulose, and wherein the composition comprises 1 to 10% (w/w) of tocopherol polyethylene glycol succinate (TPGS),

2. The composition according to claim 1, comprising:
10 to 40% (w/w) of Dabigatran etexilate;
10 to 50% (w/w) of water-soluble cellulose;
1 to 10% (w/w) of a tocopherol polyethylene glycol succinate (TPGS);
1 to 10% (w/w) of a polyoxoethylene sorbitan monooleate; and
1 to 10% (w/w) each of polyethylene glycol, glycerol and propylene glycol.

3. A composition according to claim 1, comprising:
a first layer without any solubilizer, comprising
10 to 60% (w/w) of Dabigatran etexilate,
20 to 80% (w/w) of the water-soluble cellulose, and
at least one co-solvent selected from polyethylene glycol, glycerol and propylene glycol,
a second layer without any Dabigatran etexilate, comprising
20 to 80% (w/w) of the water-soluble cellulose,
1 to 10% (w/w) of a tocopherol polyethylene glycol succinate (TPGS) as a solubilizer, and
at least one co-solvent selected from polyethylene glycol, glycerol and propylene glycol.

4. The composition according to claim 3, comprising:
a first layer without any solubilizer, comprising
10 to 60% (w/w) of Dabigatran etexilate,
20 to 80% (w/w) of the water-soluble cellulose, and
1 to 10% (w/w) each of polyethylene glycol, glycerol and propylene glycol, and
a second layer without any Dabigatran etexilate, comprising
20 to 80% (w/w) of the water-soluble cellulose,
1 to 10% (w/w) of a tocopherol polyethylene glycol succinate (TPGS),
1 to 10% (w/w) of a polyoxoethylene sorbitan monooleate, and
1 to 10% (w/w) each of polyethylene glycol, glycerol and propylene glycol.

5. The composition according to claim 1, comprising 10 to 30% (w/w) Dabigatran etexilate; 30 to 70% (w/w) water-soluble cellulose; 1 to 7% (w/w) of tocopherol polyethylene glycol succinate (TPGS); 1 to 5% (w/w) of polyoxoethylene sorbitan monooleate; 1 to 5% (w/w) polyethylene glycol; 1 to 7% (w/w) glycerol; and 1 to 7% (w/w) propylene glycol.

6. The composition according to claim 1, comprising
15 to 25% Dabigatran etexilate;
6.5% (w/w) to 7.5% (w/w) of tocopherol polyethylene glycol succinate (TPGS);
5 to 6% (w/w) polyethylene glycol;
6.5 to about 7.5% (w/w) glycerol; and
6.5 to about 7.5% (w/w) propylene glycol

7. The composition according to claim 1, wherein the water soluble cellulose is hydroxypropyl methylcellulose (HPMC).
